(19) United States
(12) Patent Application Publication
(10) Pub. No.: US 2018/0059625 A1
(43) Pub. Date: Mar. 1, 2018

(54) ALARM CLOCK IMPLEMENTATION METHOD, ALARM CLOCK IMPLEMENTATION DEVICE AND WEARABLE APPARATUS

(71) Applicant: BOE TECHNOLOGY GROUP CO., LTD., Beijing (CN)

(72) Inventors: Minqiang Yang, Beijing (CN); Hong Zhang, Beijing (CN)

(21) Appl. No.: 15/658,368

(22) Filed: Jul. 24, 2017

(30) Foreign Application Priority Data
Aug. 23, 2016 (CN) 201610709673.3

(51) Int. Cl. G04G 21/02; G04G 13/02; A61B 5/00

(52) U.S. Cl. CPC G04G 21/025; G04G 13/026; A61B 5/01; A61B 5/681; A61B 5/4809

(57) ABSTRACT

Embodiments of the present disclosure relate to the field of communication technique and provide an alarm clock implementation method, an alarm clock implementation device and a wearable apparatus including the alarm clock implementation device. The alarm clock implementation method comprises: establishing an alarm clock group containing at least two alarm clocks for one alarm clock event; and determining an alarm clock to be turned off according to a user's sleep state information and performing an OFF operation when any one of the alarm clocks in the alarm clock group except for the last alarm clock is triggered.

### CROSS-REFERENCE TO RELATED APPLICATION

This application claims the benefit of Chinese Patent Application 201610709673.3 filed on Aug. 23, 2016 in the State Intellectual Property Office of China, the whole disclosure of which is incorporated herein by reference.

### BACKGROUND OF THE INVENTION

### Field of the Invention

Embodiments of the present disclosure relate to the field of communication technique, in particular to an alarm clock implementation method, an alarm clock implementation device and a wearable apparatus including the alarm clock implementation device.

### Description of the Related Art

In daily life, many electronic devices such as mobile phones, watches, and tablets have functions to show date and time as well as an alarm clock, so that a user can set the alarm clock to achieve a reminder.

Currently, in order to avoid a situation that the user doesn't get up in time or can't be reminded due to a noisy environment, the user may consider setting a plurality of alarm clocks to perform the reminder. However, there will be some problems with doing so. Taking the alarm clock for getting up as an example, after a first alarm clock in an alarm clock group for getting up is triggered to wake up the user, if no action is performed, other alarm clocks in the alarm clock group following this first alarm clock would be triggered when a preset time is reached. Thus, it is necessary for the user to turn off other alarm clocks one by one, which brings unnecessary troubles to the user.

On the other hand, after the first alarm clock is triggered, all alarm clocks in the alarm clock group may be turned off through a confirmation operation of the user. However, considering that the user tends to continue to sleep after turning off the alarm clock, the user would not get a reminder of the subsequent alarm clocks, thereby resulting in inconvenience to the user.

### SUMMARY OF THE INVENTION

Embodiments of the present disclosure provide an alarm clock implementation method, an alarm clock implementation device and a wearable apparatus including the alarm clock implementation device to solve the defects existing in the prior arts.

Embodiments of the present disclosure are implemented by the following technical solutions.

According to one aspect of the present disclosure, there is provided an alarm clock implementation method comprising:

establishing an alarm clock group containing at least two alarm clocks for one alarm clock event; and

determining an alarm clock to be turned off according to a user's sleep state information and performing an OFF operation when any one of the alarm clocks in the alarm clock group except for the last clock alarm is triggered.

Alternatively, determining an alarm clock to be turned off according to a user's sleep state information specifically comprises:

acquiring the user's sleep state information;

determining whether the user is in an awake state according to the user's sleep state information;

determining that the current triggered alarm clock and all the un-triggered alarm clocks in the alarm clock group are the alarm clocks to be turned off if the user is in the awake state; and

determining that only the current triggered alarm clock is the alarm clock to be turned off if the user is not in the awake state.

Alternatively, acquiring the user's sleep state information specifically comprises:

acquiring user's sleep parameters in each acquisition cycle within a preset time period according to a preset acquisition cycle; and wherein the user's sleep parameters include at least one or more of a muscle relaxation, a body temperature, a pulse and a heart rate; and

obtaining the user's sleep state information by calculating weighted average of respective sleep parameters.

Alternatively, determining whether the user is in an awake state according to the user's sleep state information specifically comprises:

judging whether the user's sleep state information exceeds a predetermined threshold,

determining that the user is in the awake state if the user's sleep state information exceeds the predetermined threshold; and

determining that the user is in a sleep state if the user's sleep state information does not exceed the predetermined threshold.

Alternatively, the preset time period is less than or equals to a time interval between the current alarm clock and the next alarm clock.

According to another aspect of the present disclosure, there is provided an alarm clock implementation device comprising:

an establishing unit configured to establish an alarm clock group containing at least two alarm clocks for one alarm clock event; and

a processing unit configured to determine an alarm clock to be turned off according to a user's sleep state information and to perform an OFF operation when any one of the alarm clocks in the alarm clock group except for the last clock alarm is triggered.

Alternatively, when determining the alarm clock to be turned off according to the user's sleep state information, the processing unit is further configured to:

acquire the user's sleep state information;

determine whether the user is in an awake state according to the user's sleep state information;

determine the current triggered alarm clock and all the un-triggered alarm clocks in the alarm clock group are the alarm clocks to be turned off if the user is in the awake state; and

determine only the current triggered alarm clock is the alarm clock to be turned off if the user is not in the awake state.

Alternatively, when acquiring the user's sleep state information, the process unit is further configured to:

acquire user's sleep parameters in each acquisition cycle within a preset time period according to a preset acquisition cycle, and wherein the user's sleep parameters include at least one or more of a muscle relaxation, a body temperature, a pulse and a heart rate; and

obtain the user's sleep state information by calculating sleep parameters.

Alternatively, when determining whether the user is in an awake state according to the user's sleep state information, the process unit is further configured to:

judge whether the user's sleep state information exceeds a predetermined threshold,

determine the user is in the awake state if the user's sleep state information exceeds the predetermined threshold; and

determining that the user is in a sleep state if the user's sleep state information does not exceed the predetermined threshold.

Alternatively, the preset time period is less than or equals to a time interval between the current alarm clock and the next alarm clock.

According to a further another aspect of the present disclosure, there is provided a wearable apparatus comprising the alarm clock implementation device as described above.

### BRIEF DESCRIPTION OF THE DRAWINGS

In order to clearly explain technical solutions of embodiments of the present disclosure, the accompanying drawings necessary for describing the embodiments are briefly introduced as below. Obviously, the accompanying drawings described below are merely some embodiments of the present disclosure, and those skilled in the art will obtain other accompanying drawings according to these drawings without any inventive steps.

FIG. 1 is a schematic view of steps of an alarm clock implementation method according to an exemplary embodiment of the present disclosure.

FIG. 2 is schematic view of an alarm setting timeline according to an exemplary embodiment of the present disclosure.

FIG. 3 is a flow chart of an alarm clock implementation method according to an exemplary embodiment of the present disclosure.

FIG. 4 is a schematic view of the structure of an alarm clock implementation device according to an exemplary embodiment of the present disclosure.

### DETAILED DESCRIPTION OF PREFERRED EMBODIMENTS OF THE INVENTION

In order to make the objects, technical solutions and advantages more clear, the present disclosure will be further described in detail with reference to the accompanying drawings. It should be understood that the embodiments as described are merely a part of the embodiments of the present disclosure, rather than all embodiments thereof. According to the embodiments of the present disclosure, all other embodiments obtained by those ordinary skilled in the art without an inventive step will fall within the scope of the present disclosure.

The technical solution of the present disclosure will be described below in detail through the specific embodiments, and the present disclosure is not intended to be limited thereto.

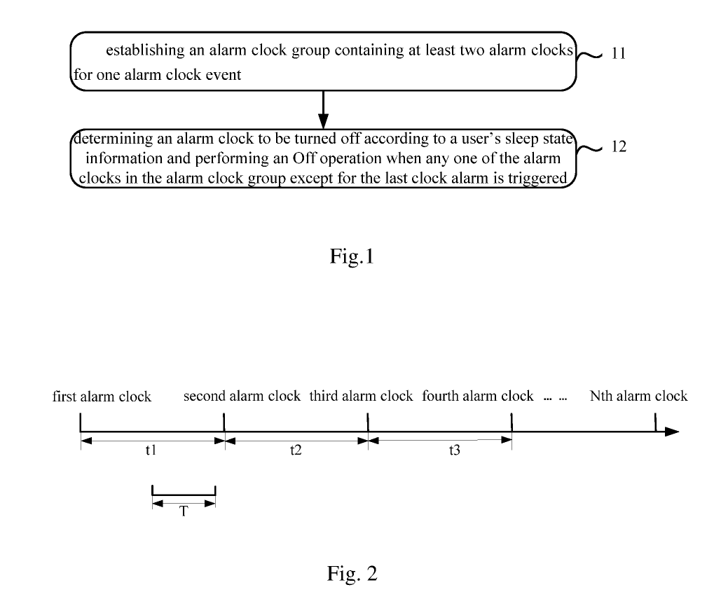

FIG. 1 is a schematic view of steps of an alarm clock implementation method according to an exemplary embodiment of the present disclosure. As shown in FIG. 1, the method is mainly implemented by an electronic device such as a wearable device, particularly by an alarm clock implementation device, which mainly comprises:

Step 11: establishing an alarm clock group containing at least two alarm clocks for one alarm clock event.

Specifically, when setting an alarm clock of the electronic device, a user may enter an alarm clock setting interface through a shortcut button or a function main interface displayed on an interactive interface of the electronic device, then an alarm setting interface is displayed on a display interface of the electronic device displays, and an alarm clock group can be set according to an operation performed through the alarm setting interface by the user, i.e., according to an alarm setting command.

It is to be noted that, in an exemplary embodiment of the present disclosure, the alarm clock group established for the electronic device may comprise a plurality of alarm clock groups. For example, an alarm clock group 1 is established for a wake-up event, an alarm clock group 2 is established for a class event, and so on. Considering importance degree of the alarm clock events to the user, an alarm clock group containing at least two alarm clocks being set chronologically may be established for any alarm clock event, so as to promptly and accurately remind the user and avoid possibility of delaying the event due to missing the reminder, thereby ensuring that the alarm clock to achieve reminder.

Step 12: determining an alarm clock to be turned off according to a user's sleep state information and performing an OFF operation when any one of the alarm clocks in the alarm clock group except for the last clock alarm is triggered.

Specifically, when any one of the alarm clocks in the alarm clock group except for the last clock alarm is triggered, an alarm clock to be turned off is determined according to a user's sleep state information and then an OFF operation is performed, rather than that all the alarm clocks are directly turned off as it does in the prior arts. Thus, in the embodiments of the present disclosure, the alarm clock to be turned off is determined by the user's sleep state information, rather than the confirmation instruction sent by the user which alarm clocks are to be turned off. In the prior art, after the alarm clock is triggered, the user needs to judge the type of the alarm clock and the time when being triggered, and then determine to sent out an instruction through a logic of mind which alarm clock should be turned off. For example, if it is judged to turn off all the alarm clocks, the button 1 is pressed; if it is judged to only turn off the current alarm clock and the next alarm clock, the button 2 is pressed, and so on. However, in the embodiments of the present disclosure, it is not necessary for the user to actively send a confirmation instruction to determine which alarm clock should be turned off. Therefore, in the embodiments of the present disclosure, there is no operation for receiving instructions sent from the user and converted into a command.

Specifically, in the step 12 of the embodiments of the present disclosure, determining an alarm clock to be turned off according to a user's sleep state information comprises:

Firstly, acquiring the user's sleep state information.

Alternatively, in the embodiments of the present disclosure, the user's sleep state information may be acquired in many ways. For example, one or more parameters characterizing the sleep state are acquired at some certain time points, and then these parameters are organized into the desired user's sleep state information through data synthesis. Specifically, during implementing the present disclosure, user's sleep parameters in each acquisition cycle are acquired within a preset time period according to a preset acquisition cycle. Then, the user's sleep state information is obtained by calculating weighted average of user's sleep parameters. The sleep parameters may include at least one or more of a muscle relaxation, a body temperature, a pulse and a heart rate. In fact, the sleep parameters may also include other human functional parameters characterizing the sleep state, and the present disclosure is not limited thereto.

Secondly, determining whether the user is in an awake state according to the user's sleep state information, and if the user is in the awake state, determining that the current triggered alarm clock and all the un-triggered alarm clocks in the alarm clock group are the alarm clocks to be turned off; otherwise, determining that only the current triggered alarm clock is the alarm clock to be turned off.

It is to be noted that, in the present disclosure, the alarm clock group is established for one alarm clock event. Further, the alarm clock implementation scheme of the present disclosure is mainly directed to the alarm clocks in each alarm clock group. Thus, when determining the alarm clock to be turned off, it is generally directed to the alarm clock group for the alarm event at the current time.

During implementing the second step, considering that the user's sleep state information is generally parameter values, it is thus possible to judge whether the user's sleep state information exceeds a predetermined threshold. If the user's sleep state information exceeds the predetermined threshold, it is determined that the user is in the awake state, and if the user's sleep state information doesn't exceed the predetermined threshold, it is determined that the user is in a sleep state. The predetermined threshold may be a parameter standard determined according to an empirical value or an individual physical fitness of the user. Further, the predetermined threshold should be obtained in the same manner as the user's sleep state information to ensure comparability among the data.

Alternatively, the preset time period involved in the embodiments of the present disclosure is equal to or less than a time interval between the current alarm clock and the next alarm clock. Specifically, as shown in FIG. 2, assuming that N alarm clocks are established for one event, i.e., a first alarm clock to a Nth alarm clock will be triggered chronologically. When the first alarm clock is triggered, it is necessary to perform the above-mentioned alarm clock implementation scheme. Thus, the preset time period is the time interval between the current alarm clock and the next alarm clock, i.e., the second alarm clock. The time interval between the first and second alarm clocks is set as t1, the time interval between the second and third alarm clocks is set as t2, and the time interval between the third and fourth alarm clocks is set as t3, and so on. The values of t1, t2 and t3 may be same or different from each other, and may be freely set according to user's habits and reminder requirements. However, regardless of the setting, when user's sleep state information is obtained for any triggered alarm clock, the preset time period should not be longer than the interval time, and each preset time period includes at least one acquisition cycle T to ensure the accuracy of the acquired user's sleep state information. Otherwise, user's sleep state information may be acquired incorrectly due to the trigger of the next alarm clock.

The above alarm clock implementation scheme will be described below in detail through an exemplary embodiment of the present disclosure.

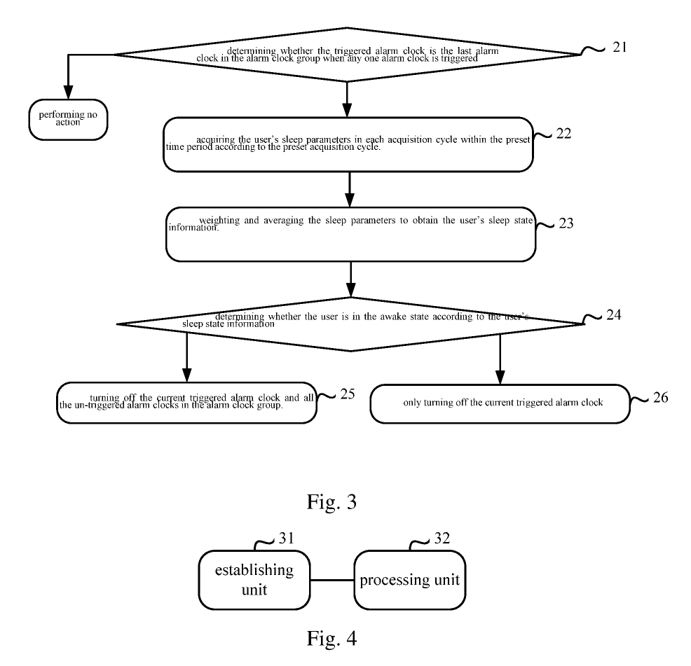

FIG. 3 shows a flow chart of an alarm clock implementation method according to an exemplary embodiment of the present disclosure. The alarm clock implementation method comprises:

S21: when any one alarm clock is triggered, determining whether the triggered alarm clock is the last alarm clock in the alarm clock group, if it is, performing no operation, otherwise performing step S22.

S22: acquiring the user's sleep parameters in each acquisition cycle within the preset time period according to the preset acquisition cycle.

S23: obtaining the user's sleep state information by calculating weighted average of respective sleep parameters.

S24: determining whether the user is in the awake state according to the user's sleep state information, and if the user is in the awake state, performing step S25; otherwise performing step S26 and turning to step S21.

S25: turning off the current triggered alarm clock and all the un-triggered alarm clocks in the alarm clock group.

S26: only turning off the current triggered alarm clock.

Thus, in the technical solution of the present disclosure, when any one of the alarm clocks in the alarm clock group except for the last alarm clock is triggered, it is possible to determine the alarm clock to be turned off according to the user's sleep state information and perform the OFF operation. Thus, it is only necessary to passively perform a functional detection through the wearable electronic device without an active operation of the user. Further, the alarm clock to be turned off is not ultimately determined by a subjective judgment of the user, but is determined through processing the information reflected by the subjective user function by the electronic device. Therefore, the adjustment flexibility of the alarm clock is improved and any confusion caused by a plurality of alarm clocks is avoided, thereby improving the user experience.

According to the same inventive concept as the alarm clock implementation method as described above, embodiments of the present disclosure further provide an alarm clock implementation device.

FIG. 4 is a schematic structural view of an alarm clock implementation device according to an exemplary embodiment of the present disclosure. The alarm clock implementation device mainly comprises an establishing unit 31 configured to establish an alarm clock group containing at least two alarm clocks for one alarm clock event, and a processing unit 32 configured to determine an alarm clock to be turned off according to a user's sleep state information and to perform an Off operation when any one of the alarm clocks in the alarm clock group except for the last clock alarm is triggered.

Alternatively, when determining the alarm clock to be turned off according to the user's sleep state information, the processing unit 32 is configured to acquire the user's sleep state information and to determine whether the user is in an awake state according to the user's sleep state information. If the user is in the awake state, the processing unit determines that the current triggered alarm clock and all the un-triggered alarm clocks in the alarm clock group are the alarm clocks to be turned off, and otherwise the processing unit determines that only the current triggered alarm clock is the alarm clock to be turned off.

It should be understood that in the disclosed embodiments, the processing unit 32 may further comprise a control subunit, an acquisition subunit and a determination subunit. The control subunit is configured to send a control signal when any one of the alarm clocks in the alarm clock group except for the last alarm clock is triggered. The control signal is output to the acquisition subunit to trigger the acquisition subunit to start a synchronization signal, that is, to start a function detection of human body, and the detected signal is transmitted to the acquisition subunit synchronously to form the desired user's sleep state information. The determination subunit is configured to determine whether the user is in the awake state according to the user's sleep state information (which may be in a signal or a instruction form) obtained by the acquisition subunit. If it is determined that the user is in the awake state, the a first OFF signal is output to the control subunit to turn off the current triggered alarm clock and all the un-triggered alarm clocks in the alarm clock group. Otherwise, a second OFF signal is output to the control subunit to turn off the current triggered alarm clock.

The control subunit may be a computer controllable modular circuit, the acquisition subunit may be a sensor circuit, and the determination subunit may be a comparison circuit comprising a diode or a transistor.

Alternatively, when acquiring the user's sleep state information, the processing unit 32 is specifically configured to acquire user's sleep parameters in each acquisition cycle within a preset time period according to a preset acquisition cycle, and the user's sleep parameters include at least one or more of a muscle relaxation, a body temperature, a pulse and a heart rate. The user's sleep state information is obtained by calculating weighted average of sleep parameters.

Alternatively, when determining whether the user is in the awake state according to the user's sleep state information, the processing unit 32 is specifically configured to judge whether the user's sleep state information exceeds a predetermined threshold, and determine the user is in the awake state if the user's sleep state information exceeds the predetermined threshold and determine the user is in a sleep state if the user's sleep state information doesn't exceed the predetermined threshold.

Alternatively, the preset time period is less than a time interval between the current alarm clock and the next alarm clock.

Furthermore, embodiments of the present disclosure further provide a wearable apparatus comprising any one of the alarm clock implementation device as described above. The wearable apparatus may include a wrist-watch, a wristband, or other wearable derives capable of contacting the skin. The wearable apparatus may be provided with a sensor device, a computer controllable device and other device capable of acquiring information on the human body function so as to control alarm clock implementation.

It should be appreciated by those skilled in the art that embodiments of the present disclosure may be provided as a method, a system or a computer program product. Therefore, the present disclosure may be implemented in form of a complete hardware, a complete software or a combination of the hardware and the software. Further, the present disclosure may be implemented in form of a computer program product carried out on one or more computer readable storage media (including but not limited to disk storage, CD-ROM, optical memory and the like) containing computer available program codes.

The present disclosure is described with reference to the flow chart and/or block diagram of a method, apparatus (system), and computer program product according to embodiments of the present disclosure. It should be understood that each process and/or block in the flow chart and/or block diagram and the combination of the processes and/or blocks in the flow chart and/or block diagram may be implemented by computer program instructions. These computer program instructions may be provided to a general purpose computer, a dedicated computer, an embedded processor, or a processor of other programmable data processing device to generate a machine to produce a device for enabling functions specified in one or more processes in the flow chart and/or one or more blocks in the block diagram through instructions executed by the computer or the processor of other programmable data processing device.

These computer program instructions may also be stored in a computer readable memory capable of operating a computer or other programmable data processing devices in a particular manner, such that the instructions stored in the computer readable memory generate a product including an instruction device implementing functions specified in one or more processes in the flow chart and/or one or more blocks in the block diagram.

These computer program instructions may also be loaded onto the computer or other programmable data processing devices such that a series of operational steps are performed in the computer or other programmable devices to produce computer-implemented processing such that the instructions executed on the computer or other programmable devices provide steps for implementing the functions specified in one or more processes in the flow chart and/or one or more blocks in the block diagram.

Although some embodiments of the present disclosure have been described, those skilled in the art can make other changes and modifications to these embodiments once reading the basic inventive concept. Thus, the appended claims are intended to be interpreted to include these embodiments and all the changes and modifications falling within the scope of the present disclosure.

Obviously, those skilled in the art may make various changes and modifications without departing from the spirit and scope of the present disclosure. Thus, if these changes and modifications of the present disclosure fall within the scope claimed in claims and their equivalents, the present disclosure is also intended to include these changes and modifications.

What is claimed is:

1. An alarm clock implementation method comprising:
establishing an alarm clock group containing at least two alarm clocks for one alarm clock event; and
determining an alarm clock to be turned off according to a user's sleep state information and performing an Off operation when any one of the alarm clocks in the alarm clock group except for a last clock alarm is triggered.

2. The method according to claim 1, wherein determining an alarm clock to be turned off according to a user's sleep state information comprises:
acquiring the user's sleep state information;
determining whether the user is in an awake state according to the user's sleep state information;
determining that a current triggered alarm clock and all un-triggered alarm clocks in the alarm clock group are the alarm clocks to be turned off if the user is in the awake state; and
determining that only the current triggered alarm clock is the alarm clock to be turned off if the user is not in the awake state.

3. The method according to claim 2, wherein acquiring the user's sleep state information comprises:
acquiring user's sleep parameters in each acquisition cycle within a preset time period according to a preset acquisition cycle, and wherein the user's sleep parameters include at least one or more of muscle relaxation, body temperature, pulse and heart rate; and
obtaining the user's sleep state information by calculating sleep parameters.

4. The method according to claim 2, wherein determining whether the user is in an awake state according to the user's sleep state information comprises:
judging whether the user's sleep state information exceeds a predetermined threshold;
determining that the user is in the awake state if the user's sleep state information exceeds the predetermined threshold; and
determining that the user is in a sleep state if the user's sleep state information does not exceed the predetermined threshold.

5. The method according to claim 3, wherein the preset time period is less than or equal to a time interval between the current alarm clock and a next alarm clock.

6. An alarm clock implementation device comprising:
an establishing unit configured to establish an alarm clock group containing at least two alarm clocks for one alarm clock event; and
a processing unit configured to determine an alarm clock to be turned off according to a user's sleep state information and to perform an OFF operation when any one of the alarm clocks in the alarm clock group except for a last clock alarm is triggered.

7. The device according to claim 6, wherein, when determining the alarm clock to be turned off according to the user's sleep state information, the processing unit is further configured to:
acquire the user's sleep state information;
determine whether the user is in an awake state according to the user's sleep state information;
determine a current triggered alarm clock and all un-triggered alarm clocks in the alarm clock group are the alarm clocks to be turned off if the user is in the awake state; and
determine only the current triggered alarm clock is the alarm clock to be turned off if the user is not in the awake state.

8. The device according to claim 7, wherein, when acquiring the user's sleep state information, the process unit is further configured to:
acquire user's sleep parameters in each acquisition cycle within a preset time period according to a preset acquisition cycle, and wherein the user's sleep parameters include at least one or more of muscle relaxation, body temperature, pulse and heart rate; and
obtain the user's sleep state information by calculating a weighted average of sleep parameters.

9. The device according to claim 7, wherein, when determining whether the user is in an awake state according to the user's sleep state information, the process unit is further configured to:
judge whether the user's sleep state information exceeds a predetermined threshold,
determine the user is in the awake state if the user's sleep state information exceeds the predetermined threshold; and
determine that the user is in a sleep state if the user's sleep state information does not exceed the predetermined threshold.

10. The device according to claim 8, wherein the preset time period is less than or equal to a time interval between the current alarm clock and a next alarm clock.

11. A wearable apparatus comprising the alarm clock implementation device according to claim 6.
